(19) United States
(12) Patent Application Publication
(10) Pub. No.: US 2017/0253850 A1
(43) Pub. Date: Sep. 7, 2017

(54) Carbon Neutral Composting Achieved by Cooperative Aerobic Composting and Algae Cultivation

(71) Applicant: Phillip Marc Ranglin, Decatur, GA (US)

(72) Inventor: Phillip Marc Ranglin, Decatur, GA (US)

(21) Appl. No.: 15/058,019

(22) Filed: Mar. 1, 2016

(51) Int. Cl. C12N 1/12; C12F 3/02

(52) U.S. Cl. CPC C12N 1/12; C12F 3/02

(57) ABSTRACT

Organic waste disposal in landfills is a substantial source of methane and carbon dioxide. Composting is an excellent alternative to landfill use, and the process described herein demonstrates a method to use such source-separated organics as a Carbon dioxide source for industrial scale algae cultivation, functionally sequestering the greenhouse gases produced.

Carbon neutral composting is achieved by the activity of microscopic autotrophs, algae, which trap carbon dioxide, CO₂, as population expansion occurs. This organism is doubly beneficial, in that they store excess energy produced as oil pockets within individual cells. This oil is a perfect raw material for biodiesel production, as its chemical composition is both predictable and consistent, allowing engineers to manufacture engines more capable of using this advanced biofuel.

The invention described herein marries algae cultivation to the aerobic breakdown of source-separated organic materials, simultaneously achieving the sequestration of carbon. A two circuit system facilitates this process: the gaseous circuit uses negative pressure to extract CO₂ rich effluent air from the compost chamber, providing multiple opportunities to maximally dissolve this gas, making it available to growing algal populations; the algae-containing liquid circuit connects and circulates the common medium from one end of the series of chambers to the other, by use of a diaphragm pump. A potential difference is created when liquid from the first container is transferred to a photo bioreactor, then is deposited into the terminal vessel, with bulkhead connections between the containers allowing unrestricted flow of fluid.

### BACKGROUND OF THE INVENTION

Unrestricted gaseous carbon release, to the tune of 40 billion annual tons, has become a chronic problem for mankind. Under normal circumstances, carbon dioxide is released and shortly thereafter becomes trapped as a photosynthetic raw material by autotrophic organisms, producing energy by using sunlight and a carbon source. A careful balance must be maintained to keep atmospheric temperatures within a habitable range, which is compromised by human activities such as deforestation, leaving a surplus of gaseous carbon that traps heat and causes global warming.

Methane has also become problematic, due to agriculture and landfill production, the latter of which is more important in the context of this new process design.

This invention falls into the field of the biological sciences, but has far reaching implications into the Geological sciences, Chemistry and Engineering.

Algae cultivation, the inspiration of this invention, is a way to use a smaller carbon loop for energy production, reducing the demand for the massive carbon sequestration unit, crude oil. If gaseous carbon sources had an avenue within which to undergo the high temperature and pressure cycling that resulted in crude oil creation, climate change would not be an issue. However, a temporal bottleneck has occurred because crude oil takes millennia to regenerate, yet only milliseconds to combust when used as fuel. The impact of this is compounded by rapid deforestation and the continued, limitless use of fossil fuels to power human activities. Algae, however, represent a possible solution—an organism that can trap atmospheric CO₂ at a rapid rate (due to its short doubling time). Once in high density, algae can store oil, a perfect raw material for biodiesel production, or whole cell biomass can be treated with high heat and pressure to produce a renewable product similar to crude oil, which can then be distilled into many of the original fractions. However, atmospheric CO₂ levels, although high enough to cause global warming, are not high enough to be conducive to large scale algal cultivation at the level needed to realistically replace or even supplement current fuel sources.

Within these data are several problems:

1. Algae cultivation and oil production are a straightforward way to mitigate fossil fuel demand, and is a much cleaner operation. However, current industrial scale operations utilize processed sugars and glycerol as carbon sources, completely ignoring the overwhelming benefit capability of this organism in its ability to trap gaseous CO₂. The first question, therefore, is, how does one provide higher concentrations of CO₂ than levels within ambient air, in a more sustainable manner?

2. Landfills are utilized worldwide as a general destination for solid waste. However, these spaces create anaerobic environments, which encourages the production of methane and CO₂, the former trapping more than 70 times the heat of its counterpart. Furthermore, gas collected from landfills is laden with carcinogenic and otherwise toxic components, which may become more harmful when this gas is used as a fuel for internal combustion engines.

3. With dwindling fossil fuel reserves and increasingly dangerous and toxic extraction methods being developed, in conjunction with massive, widespread deforestation, how can a fuel source with a smaller carbon footprint be integrated into the current infrastructure?

### BRIEF SUMMARY

Aerobic composting partially answers all 3 questions, with the invention of focus making up the difference. The first benefit of composting is in its ability to divert source separated organic materials from landfills, which reduces the need for additional sites, and diminishes the amount of substrate availability for anaerobic respiration (and, hence, methane production).

This invention is carbon neutral because it is designed to maximally trap CO₂ produced in aerobic breakdown of organic materials. The carbon sequestered in this process eventually becomes oil or biomass, which can be converted into bio-crude or biodiesel, respectively.

Additionally, as organics are diverted and carbon sequestered as solid or liquid states of matter, mitigation of the atmospheric imbalance becomes less impossible.

This invention solves the primary problem of responsibly sourcing carbon dioxide for industrial algae cultivation, simultaneously diverting organic waste from landfills in the process. Gaseous carbon produced as organic materials decompose is trapped, the cultivation medium acting as a scrub—dissolving the gas by offering multiple opportunities for dissolution. The gas is separated from pass to another by use of separate chambers with connections only below the liquid level, or having a septum separating each passage of the air.

### BRIEF DESCRIPTION OF THE SEVERAL VIEWS OF THE DRAWING

FIG. 1 illustrates a schematic view of the entire setup. Ambient air enters the system at 1, passes through the composting chamber, then gets introduced into the repeated portion of the design, within the brackets. The singular liquid circuit output to the photobioreactor is also shown, which goes to the terminal vessel for passive volume equilibration.

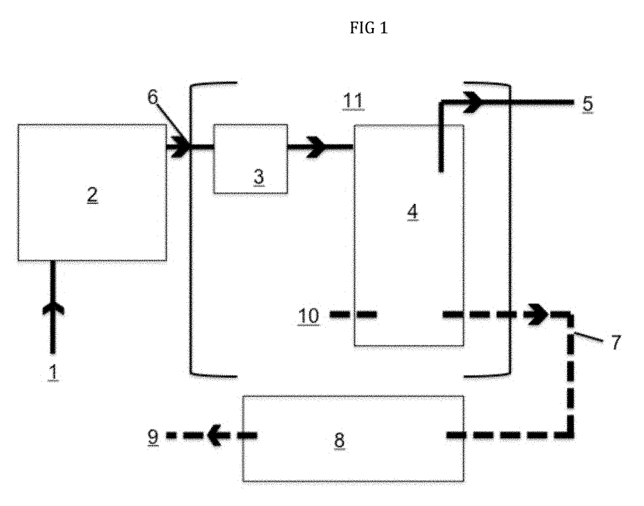

FIG. 2 shows a detailed view of a single unit of the carbon neutral composting system. This unit may be repeated as many times as necessary, and the output labeled 20 can be either the final output to ambient air, or it can supply another pump housing, 14.

### DETAILED DESCRIPTION

The beauty of this method of carbon sourcing for algae cultivation is in its simplicity. All processes are natural and require no genetic modification. The size of each component can be scaled up or down, based on need, allowing universal application of the design. The process is simple—aerobic composting (such as earthworm or microbial) breaks down consumer separated organic material, enriches air with carbon dioxide, then passes this air through an algal cultivation medium. The use of the carbon dioxide source is the first signature portion of this patent, the second being the multiple passages of air through the same cultivation medium, facilitating maximal dissolution of this greenhouse gas. The cultivation medium is connected by bulkhead connectors, and each vessel corresponds to a dissolution cycle.

To understand the main portion of this process, one may examine the route taken by air through the system.

Air enters the system through pores in the compost vessel, 12, lured by a transient vacuum. It then passes through spaces in the compost medium, becoming enriched as it resolves the upstream negative pressure. Upon emerging from this metabolically active layer, 13, the air passes through tubing to the source of the vacuum, a pump within an airtight enclosure, 14. As air is pumped from the sealed enclosure, it is replenished by effluent gas from the downstream compost container. The carbon dioxide rich effluent gas now passes from the pump housing to the first dissolution point, via the manifold, 16, for the corresponding vessel, 15. Ceramic air stones generate very fine bubbles, which promote carbon dioxide dissolution into the medium, 17, as the air floats to the surface of the sealed vessel. An outward flowing exit point for this air, 18, (maintained by a check valve) permits the passage or the air from the first tank to the pump housing of the second, after which it again passes through a second manifold. This process may be repeated 2-5 times to allow maximum dissolution of carbon dioxide gas, with the concentration decreasing with each passage. At the end of the process, the outward-flowing check valve goes to external air, 20, with an in-line filter present to reduce contamination.

Within the liquid circuit, algae cultivation medium composition depends on the strain being used, as do the intensity and duration of light. The liquid can be circulated according to the requisite scale. For demonstration purposes, two 55 gallon drums with sealable lids were used, and a 1 inch bulkhead fitting collected the barrels (21, 23) ½ inch tubing collects culture medium, and a diaphragm pump (8, 22) connected to an air compressor circulated the liquid, passively returning it to the barrel at the end of the series. Bulkhead connections from one to the next allow passive equalization of volume, and contribute to circulation of medium, while maintaining uniformity of ingredient distribution.

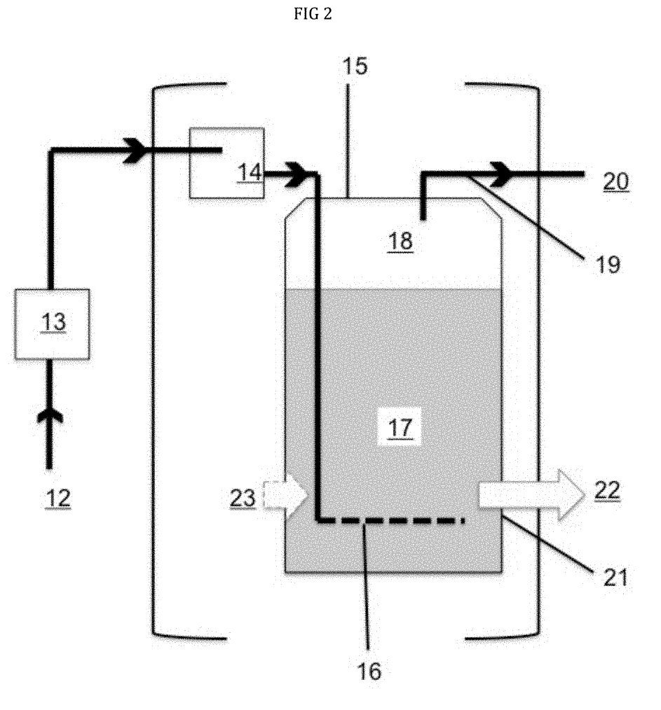

FIG. 2 represents the patent-relevant portion. Source-separated organics (13) are broken down aerobically, as ambient air passes through the system (12). Pump 14 draws air from the compost chamber (13), using it to aerate the first vessel, 15, as CO₂ dissolves in medium 17, compost effluent air collects at the peak of the vessel, 18. This air is then used to restart the bracketed cycle, as tube 19 connects to pump 14 of a subsequent vessel. Liquid is fully connected between the vessels, by bulkhead connectors 22 and 23. Output 22 goes to the photobioreactor setup (not shown), and terminates in the final vessel (23 of final vessel), creating a downward current, which further assists in circulation of nutrients.

Harvesting intervals will depend on the scale of the operation. Once the air input originates at a composting vessel, and is used to aerate one tank, then the same quantity of air is circulated through subsequent tanks, the goal of CO₂ dissolution is achieved, and the premise of this process design is in use.

1. The process uses a multiple aeration chamber to repeatedly circulate CO₂ rich effluent from aerobic composters, sequestering the majority of carbon, as CO₂, in the process described below:
a. The aerobic metabolic pathway is promoted by continuous flow of fresh air into the cultivation medium, be that containing earthworms (vermicomposting) or a stand-alone microbial milieu.
b. The carbon dioxide produced during this process is used as a photosynthetic substrate for algae growth and population expansion.
c. Negative pressure is generated by the use of an air compressor (continuous pump or air compressor, dependent upon scale of use), which is sealed in a container with an out port, maintaining one-way airflow (pulling from composting chamber) via the fitment of check valves.
d. Carbon Dioxide concentration is reduced by multiple dissolution cycles, wherein micro aerators bubble air through the cultivation medium, generating a large surface area for dissolution. In the first such cycle, the compost container provides the effluent gas, rich in the photosynthetic substrate. This air is bubbled through the cultivation medium, and, when it rises to the top of the sealed vessel, acts as the source for the next unit in the series (supplying the sealed compressor chamber), allowing the process to be repeated, and so on. At the end of several cycles (depending on CO₂ content), the terminal vessel empties into ambient air.
e. The liquid medium must be connected from one vessel to another, to keep the nutrient concentrations consistent, and to facilitate dissolution of gases.
f. Extracted culture medium at the beginning of the series of vessels is treated with species-specific illumination, and returned to the terminal container. Gravity will balance the liquid levels, and this will also ensure proper mixing of solutes.
